(19) United States
(12) Patent Application Publication
(10) Pub. No.: US 2017/0007188 A1
(43) Pub. Date: Jan. 12, 2017

(54) Extracting Ventricular Ejection Fraction from Pressure Sensing Data

(71) Applicants: Robert W. Biederman, Wexford, PA (US); Raymond L. Benza, Pittsburgh, PA (US); Mark Doyle, Wexford, PA (US)

(72) Inventors: Robert W. Biederman, Wexford, PA (US); Raymond L. Benza, Pittsburgh, PA (US); Mark Doyle, Wexford, PA (US)

(21) Appl. No.: 15/207,174

(22) Filed: Jul. 11, 2016

Related U.S. Application Data
(60) Provisional application No. 62/190,827, filed on Jul. 10, 2015.

(51) Int. Cl. A61B 5/00; A61B 5/026; A61B 5/029; A61B 5/145; A61B 5/021; A61B 5/02

(52) U.S. Cl. CPC A61B 5/7278; A61B 5/021; A61B 5/02028; A61B 5/029; A61B 5/7225; A61B 5/14542; A61B 5/4866; A61B 5/4884; A61B 5/0263; A61B 2560/0223

(57) ABSTRACT

A method of and system for determining ventricular ejection fraction of a patient is provided. A pressure sensing device captures pulmonary arterial pressure data for a patient over time. A processing device receives the pressure data, generates a first time-resolved pressure curve, displaces the pressure values of the first time-resolved curve at least one time point and subtracts the displaced pressure values from the received pressure data to form a second time-resolved pressure curve so that the second curve has two or more distinct pulses from which an initial pulse may be isolated and an area may be calculated. The processing device determines an average pressure by averaging the pressure data of the first curve over a cardiac cycle of data; determines a cardiac chamber stroke volume for the patient; and uses the determined cardiac chamber stroke volume and determined average pressure to determine an ejection fraction for the patient.

### RELATED APPLICATIONS AND CLAIM OF PRIORITY

This patent document claims priority to U.S. Provisional Patent Application No. 62/190,827, filed Jul. 10, 2015, titled “Extracting Ventricular Ejection Fraction from Pressure Sensing Data.” The disclosure of the priority application is fully incorporated into this document by reference.

### STATEMENT REGARDING FEDERALLY SPONSORED RESEARCH

This invention was made with government support under Contract Number HHSN268201400008C awarded by the National Institutes of Health. The government has certain rights in the invention.

### BACKGROUND

Pressure sensing devices are commonly used in evaluation of patients with cardiovascular disease. Such devices include a pressure sensing catheter and a micro-manometer device that is implanted in the lumen of the pulmonary artery and senses pressure in the pulmonary artery of patients with pulmonary hypertension. When used in patients with pulmonary artery hypertension, such devices, in combination with cardiac magnetic resonance imaging (MRI) devices, can report the pulmonary blood pressure and yield data that can be used to extract the right ventricular stroke volume from the pressure curve. However, the reported stroke volume can be inaccurate in current approaches.

Ejection fraction (EF) is a measurement of how much blood a ventricle pumps out with each contraction. EF values can be used to assess how well a patient's heart is operating, and can help diagnose heart failure and risks of potential heart failure. For example, a heart's normal EF may be considered to be between 50 and 70. An EF that is under 40 may be considered to be evidence of heart failure, while an EF that is between 41 to 49 may indicate that a patient is at risk of heart failure, or that the patient's heart has been damaged in the past by a heart attack or other incident. Current methods of determining EF can also be inaccurate.

Therefore what is needed are new methods and systems for determining stroke volume and EF that improve on the current methods. The systems and processes described in this document can be used to more accurately diagnose heart failures and identify patients who are at risk of heart failure.

### SUMMARY

The foregoing needs are addressed by the system and method disclosed herein that uses knowledge of the stroke volume to calculate the ejection fraction of the right ventricle.

This document identifies an approach that extracts the stroke volume from the pressure curve yielded from a pressure sensing catheter applied to a patient with greater accuracy following a normalization using cardiac MRI data. Further, this document also describes an approach to extract the right ventricular ejection fraction (EF) from the pressure curve, representing an additional and novel step. The principles described below can also be applied to cardiac catheter data.

In one aspect, a method of determining ventricular ejection fraction of a patient is provided. The method includes providing a pressure sensing device for capturing pulmonary arterial pressure data for a patient over a period of time; and providing a processing device for implementing programming instructions that are configured to cause the processing device to receive the pressure data captured by the pressure sensing device.

In another aspect, the processing device generates a first time-resolved pressure curve that includes the pressure data; displaces the pressure values of the first time-resolved curve at least one time point and subtracts the displaced pressure values from the received pressure data to form a second time-resolved pressure curve so that the second time-resolved pressure curve has two or more distinct pulses from which an initial pulse may be isolated and an area may be calculated.

In another aspect the processing device determines an average pressure by averaging the pressure data of the first time-resolved pressure curve over a cardiac cycle of data; determines a cardiac chamber stroke volume for the patient; uses the determined cardiac chamber stroke volume and determined average pressure to determine an ejection fraction for the patient; and outputs a report of the ejection fraction

In another aspect, determining the ejection fraction is also based on a slope of a rise in pressure during systolic contraction and the average pressure.

In another aspect, determining the ejection fraction comprises applying the following equation:

EF=(SV*Emax)/(mean P*(ΔP+SV*ΔP));
wherein:
EF is the ejection fraction,
SV is the stroke volume,
Emax is a slope of a rise in pressure during systolic contraction,
mean P is the average pressure, and
ΔP is a difference between an end systolic pressure and an end diastolic pressure as determined in the pressure data.

In another aspect, determining the stroke volume includes determining an area under a first pulse of the first curve; multiplying the area by a constant to yield a result; and dividing the result by the average pressure.

In another aspect, the processing device performs a calibration step by using data received from an imaging modality, a flow based measurement, or other measurement means to measure a cardiac chamber stroke volume, and uses the measured cardiac chamber stroke volume to calculate the constant.

In another aspect, the constant is 2.37.

In another aspect, the processing device implements programming instructions that are configured to cause the processing device to determine an additional cardiac chamber stroke volume for an additional time period using an additional pressure waveform for the additional time period, and the constant; and use the additional cardiac chamber stroke volume to determine an additional ejection fraction of the patient.

In another aspect, determining the cardiac chamber stroke volume comprises calculating the difference in oxygen concentration between an arterial and venous blood supply and a total oxygen consumption per minute.

In another aspect, determining the cardiac chamber stroke volume comprises injecting a measured volume of cooled liquid at a measured temperature into a right atrium; and calculating a carbon monoxide level by an amount of heat lost.

In another aspect, a system for determining ventricular ejection fraction of a patient is provided. The system includes a pressure sensing device for capturing pulmonary arterial pressure data for a patient over a period of time; and a processing device for implementing programming instructions.

In another aspect, the processing device receives the pressure data captured by the pressure sensing device; generates a first time-resolved pressure curve that comprises the pressure data; displaces the pressure values of the first time-resolved curve at least one time point and subtract the displaced pressure values from the received pressure data to form a second time-resolved pressure curve so that the second time-resolved pressure curve has two or more distinct pulses from which an initial pulse may be isolated and an area may be calculated; determines an average pressure by averaging the pressure data of the first time-resolved pressure curve over a cardiac cycle of data; determines a cardiac chamber stroke volume for the patient; uses the determined cardiac chamber stroke volume and determined average pressure to determine an ejection fraction for the patient; and outputs a report of the ejection fraction.

These and other features and aspects of the invention will now be described with reference to the accompanying Figures and the Detailed Description.

### BRIEF DESCRIPTION OF THE DRAWINGS

FIG. 1 is a flow diagram illustrating a process of determining ejection fraction for a patient.

FIG. 2 illustrates an example of a pressure waveform that may be extracted using the embodiments described below.

FIG. 3 illustrates an example of a time differential that may be determined using the embodiments described below.

FIG. 4 illustrates an example output using the embodiments described below.

FIG. 5 illustrates an example output of prior art methods.

FIGS. 6-9 illustrate example outputs using the embodiments described below

FIG. 10 illustrates example components of a computing device.

### DETAILED DESCRIPTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.”

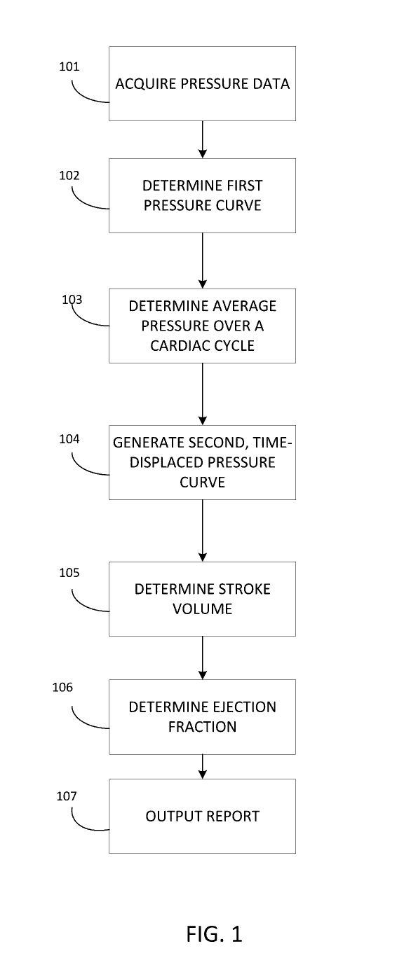

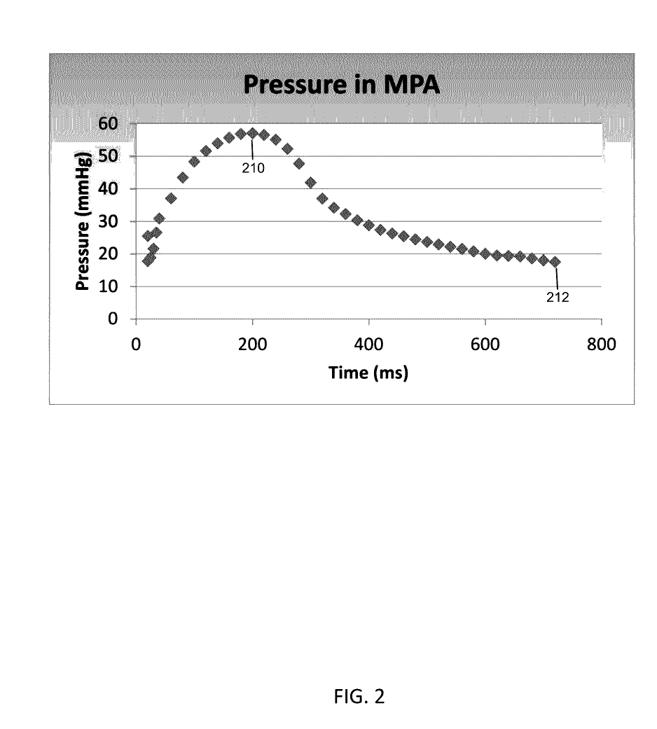

An example method of determining a patient's ejection fraction (EF) is disclosed hereinbelow. Referring to FIG. 1, data representing measurements of the pressure of a patient's pulmonary artery (PA) is obtained over a period of time 101 using an intercorporeal pressure sensor. An example of such data is shown in FIG. 2, which is a waveform generated with data obtained via a micro-manometer implanted in the main pulmonary artery of a patient with pulmonary hypertension. In FIG. 2, pressure is shown in mmHG over time in milliseconds (ms), although other values can be used. This data may be captured by placing a miniaturized, wireless monitoring sensor pressure sensing device in a vascular lumen, such as the pulmonary artery or cardiac chamber, of a patient during a minimally invasive procedure to directly measure pressure. For example, measuring pulmonary arterial pressure allows clinicians to proactively manage treatment for patients with worsening heart failure before visible symptoms, such as weight and blood pressure changes, occur. The implantable sensor/monitor may comprise a battery-free capacitive pressure sensor permanently or removably implanted in the pulmonary artery or cardiac chamber by a transvenous delivery system designed to deploy the implantable sensor in the distal pulmonary artery or cardiac chamber. An electronics system, known to those of skill in the art, acquires and processes signals from the implantable sensor/monitor and transfers systolic and diastolic pressure measurements to a secure database. The implantable sensor may utilize an inductive-capacitive (“LC”) resonant circuit with a variable capacitor. The capacitance of the circuit varies with the pressure of the environment in which the sensor is located and thus, the resonant frequency of the circuit varies as the pressure varies. As a result, the resonant frequency of the circuit may be used to calculate systolic and diastolic pressure.

It is known that the spatial pressure differential is responsible for driving flow. However, this information is not available using existing pressure sensing catheters. Further, in the pulmonary artery vasculature the pressure waveform is quickly contaminated by reflected waves.

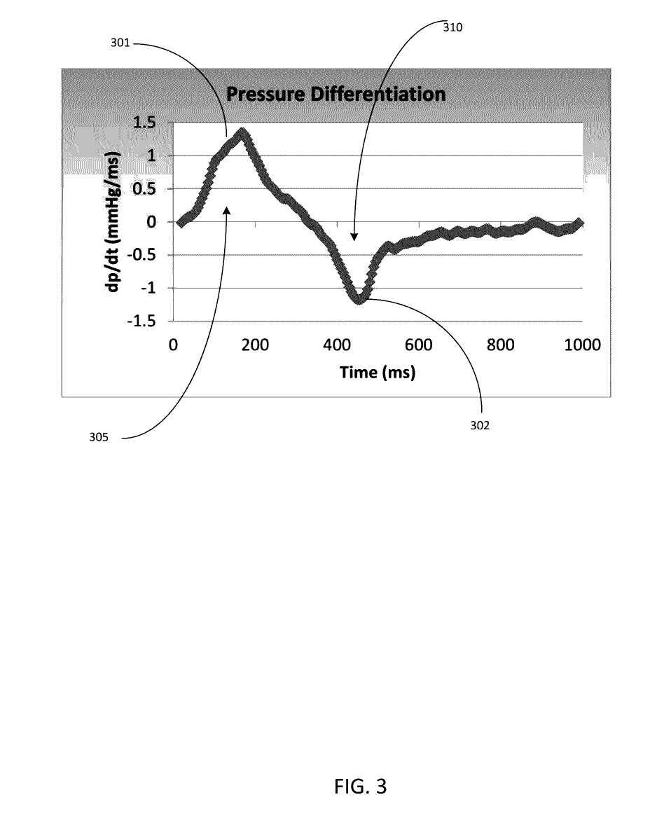

To help address this issue, this document describes a solution that extracts the pressure curve (waveform) from the pressure sensing device 102 (see also FIG. 2), and that calculates an average pressure of the values in the first pressure curve over a complete or substantially complete cardiac cycle of data 103. The system also may generate a second, time-resolved pressure curve 104 based on the first curve and a time differential (see also FIG. 3). The system then determines a stroke volume 105 for the patient using any suitable method, such as by calculating an area under a first pulse of the time differential curve (i.e., the second, time-displaced curve of FIG. 3). It then uses the stroke volume and other data as described below to determine an ejection fraction 106 for the patient. The system will output a report 107 of the ejection fraction, such as by displaying it on a display, printing it on a substrate, outputting it in audio form, or saving it to a computer-readable memory.

To calculate the pressure differential curve (FIG. 3), the initial time-resolved pressure waveform is stored in a computer-readable memory. In this embodiment, the x-axis represents time and the y-axis represents pressure. The system generates the second time-resolved pressure curve by displacing the original time-resolved pressure curve in the positive time direction by at least one time point. The two data sets are subtracted, and typically exhibit two to three distinct pulses over the cardiac cycle.

To determine a stroke volume, the system may multiply the value of an area under a first pulse of the second (displaced) curve by a constant, and divide the result by the average pressure of the first curve. The initial (positive) pulse 301 is identified as the first lobe of positive values, and the area under the pulse 305 (i.e., between the pulse and the zero line of the y-axis) may be calculated, such as by using a sum of the pressure values. The negative time differential does not need to be calculated, since the late retarding pressure differential has diminished influence. However, the embodiments in this document do not exclude calculation of a negative time differential. To a first order the positive area is taken as the net driving pressure for flow and the peripheral resistance that opposes the forward flow is directly related to the mean pressure. Thus the net pulsatile forward flow is proportional to the pressure differential of a first peak area divided by the mean pressure. When this document uses the term “first” or “initial” and “under,” it is intended that any pulse, such as second pulse 302 and an area over the pulse 310 (i.e., between the pulse and the zero on the y-axis), will be included in such terms.

The constant of proportionality may be found using a known cardiac stroke volume in a calibration step. For example, in the case of the implanted micro-manometer device, one may use a magnetic resonance imaging (MRI)-derived stroke volume.

In an alternate approach, other methods of determining stroke volume may be used. Such methods may include those known to those of skill in the art as the Fick method or the dilution method. In the Fick method, the difference in oxygen concentration between the arterial and venous blood supply and the total oxygen consumption per minute is used to calculate cardiac output. In the dilution method, a measured volume of cooled liquid (at a measured temperature) is injected into the right atrium and is sensed downstream by a thermistor. A computer is typically used to integrate this time-resolved information and calculate the carbon monoxide (CO) by the amount of heat lost.

### Example

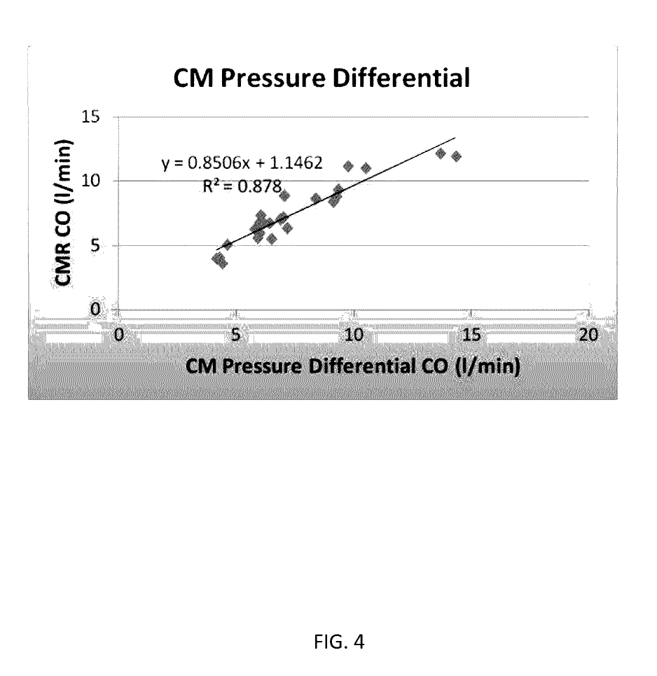

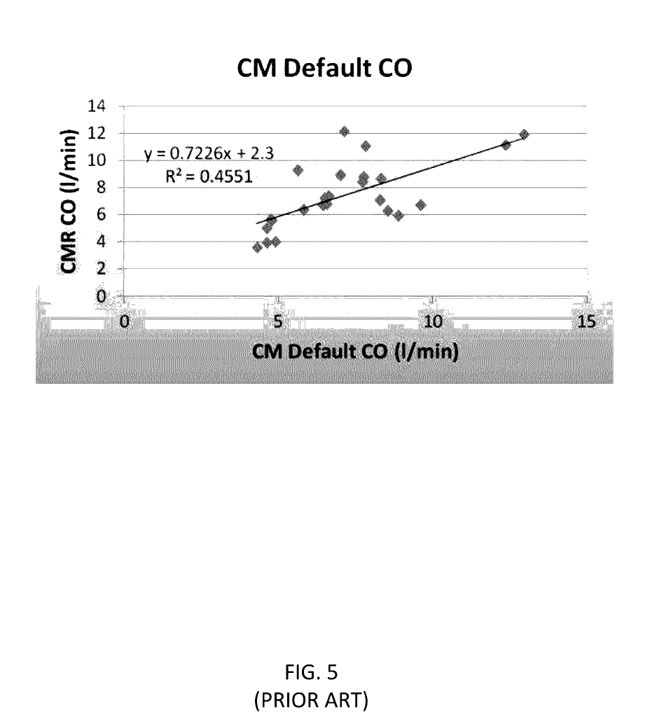

An analysis was performed for six patients and used to predict the cardiac output (stroke volume×heart rate) for several stress situations, FIG. 4. This was more accurate than the previous calculation of cardiac output using prior art methods, an example calculation of which is shown in FIG. 5.

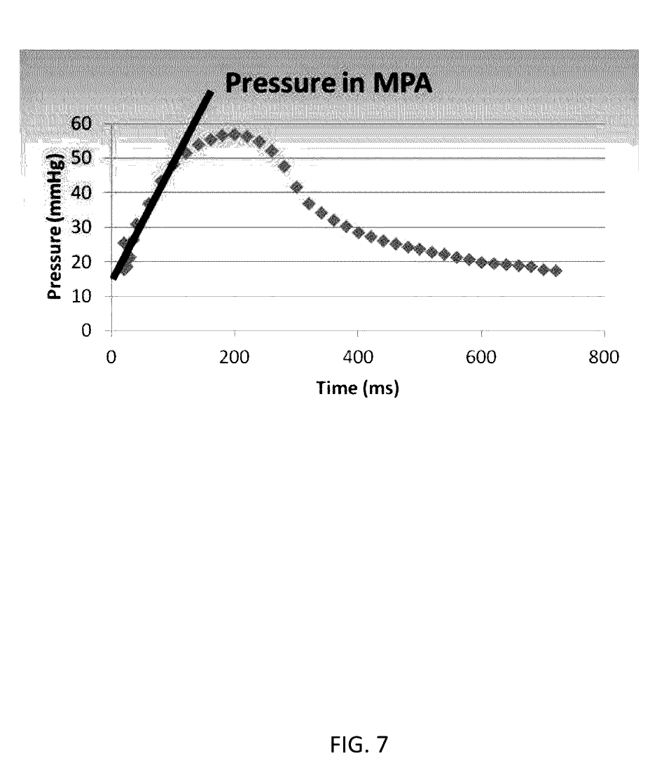

Method of calculation of Ejection Fraction (EF): With reference to the pressure waveform, pressure temporal differentiation curve, and cardiac energetics pressure-volume loop, (examples of which are shown in in FIGS. 2, 3, 6 and 7, respectively), the system may determine EF using the following algorithms:

EF=SV/EDV (1)

Tan α=(ESP−EDP)/(EDV−SV) (2)

EDV=(ΔP+SV*ΔP)/Tan α (3)

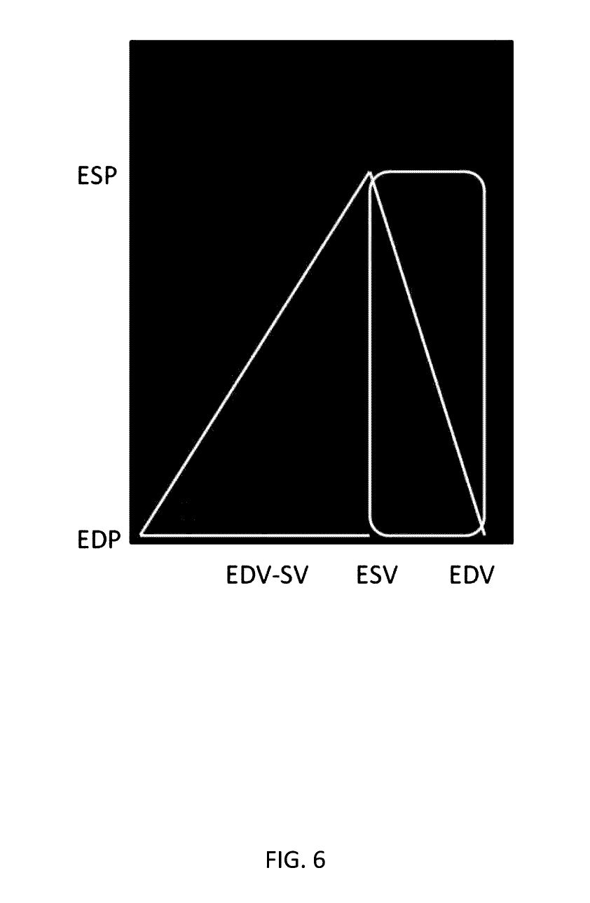

Tan α=Emax (4)

Where CMR-measured parameters are: EF is the ejection fraction, SV is the stroke volume of the right ventricle, EDV is the end diastolic volume of the right ventricle, ESV is the end systolic volume of the right ventricle, and micro-manometer-measured parameters are: ESP is the end systolic pressure 210, EDP is the end diastolic pressure 212, ΔP is the ESP-EDP pressure difference, and composite parameters derived from the combination of CMR and micro-manometer parameters as indicated in FIG. 6 and: α the angle defined by the ESP-EDP-ESV which is defined Emax which is the maximum elastance of the myocardium during cardiac ejection. In other words, Emax is the slope of the angle shown in FIG. 6. While this document uses examples that refer to the right ventricle, those of ordinary skill in the art will appreciate that the embodiments described in this document equally apply to calculation of left ventricle ejection fraction.

Thus, equating equations (2) and (4) above leads to an expression of EF in terms of Emax:

EF equals(SV*Emax)/(mean P*(ΔP+SV*ΔP)) (5)

To derive the forgoing, EF was measured using MRI volumetric imaging, by taking serial images through the heart and outlining the endocardial boundary of the left ventricle at end diastole and at end systole. From these measures the end diastolic and end systolic volumes of the left ventricle were determined. The EF was then calculated using the standard formula.

EF=(end diastolic volume−end systolic volume)/(end diastolic volume).

This measure of EF was used experimentally to relate to the pressure derived variables. Thus the relationship of the pressure derived variables to EF was found in this manner.

The initial upslope of the pressure-time curve is related to Emax, thus as noted above the up slope of the pressure curve during a systolic contraction was taken to represent Emax in equation (5), FIG. 6. In equation 5, “mean P” is the determined average pressure from the first pressure curve, as described above. Thus, in operation the system may use equation (5) such that EF=(SV*Emax)/(mean P*(ΔP+SV*ΔP))

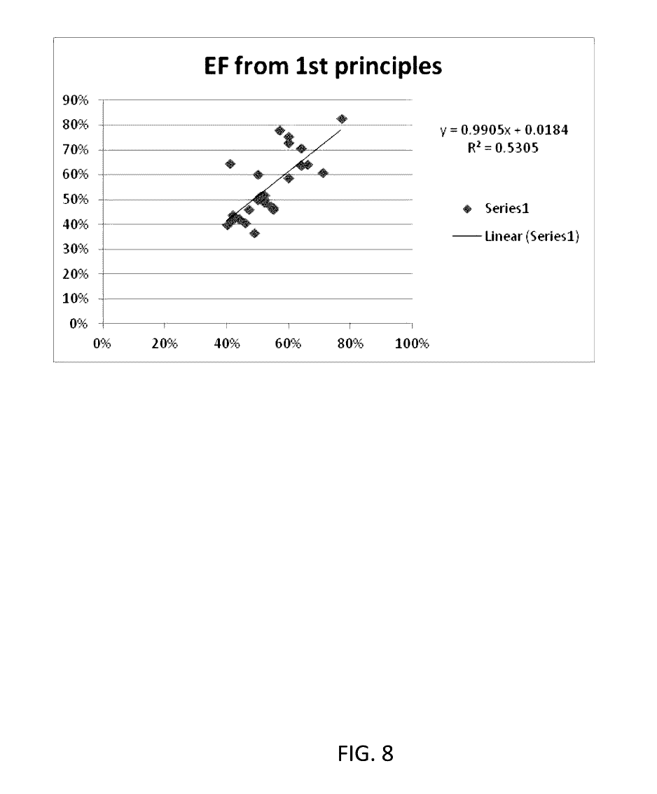

For with a pressure sensing device implanted, the pressure curve may be used to calculate the SV and ΔP, and the maximum initial upslope and substituted in equation (5). To calibrate the system, the system may acquire cardiac MRI (CMRI) data to find a constant of proportionality for each patient using the determined stroke volume, and to allow the calculation of EF for subsequent stress conditions, FIG. 8. Other methods of determining stroke volume may be used in the calibration step, such as a flow based measurement or data from another imaging modality. When MRI volumetric imaging is used for calibration, the system may take serial images through the heart and outlining the endocardial boundary of the right ventricle at end diastole and at end systole. From these measures the EDV and ESV of the right ventricle may be determined. The EF may then be calculated using the standard formula EF=(EDV−ESV)/EDV, and the measure may be used to derive the pressure derived variables, such as the constant.

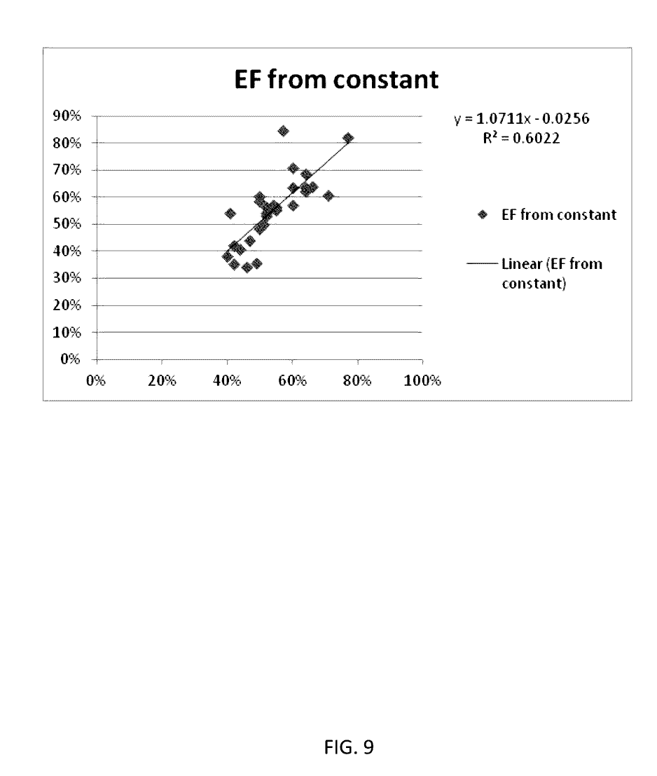

Thus, in various embodiments a value of EF may be known for at least one point for each patient. In a test involving six patients, the inventors surprisingly observed that the constant of proportionality was similar for each patient. Thus, the system may initially calculate the SV for each patient from the pressure data using methods described above with a constant, and then using the algorithms described above to determine EF based on the SV. In various embodiments, the constant may be 2.37 (see FIG. 9). In other embodiments, the constant may be within +/−5% of 2.37 (i.e., 2.25 to 2.49), +/−10% of 2.37 (i.e., 2.13 to 2.61), or +/−20% of 2.37 (i.e., 1.896 to 2.844. Other values are possible. Thus, to calculate EF it is only required to calibrate each patient using once for stroke volume.

In some embodiments, once calibration has been performed, the system may determine an additional cardiac chamber stroke volume for an additional time period using the constant along with an additional pressure waveform for the additional time period and the constant. The system may then use the additional cardiac chamber stroke volume to determine an additional ejection fraction of the patient.

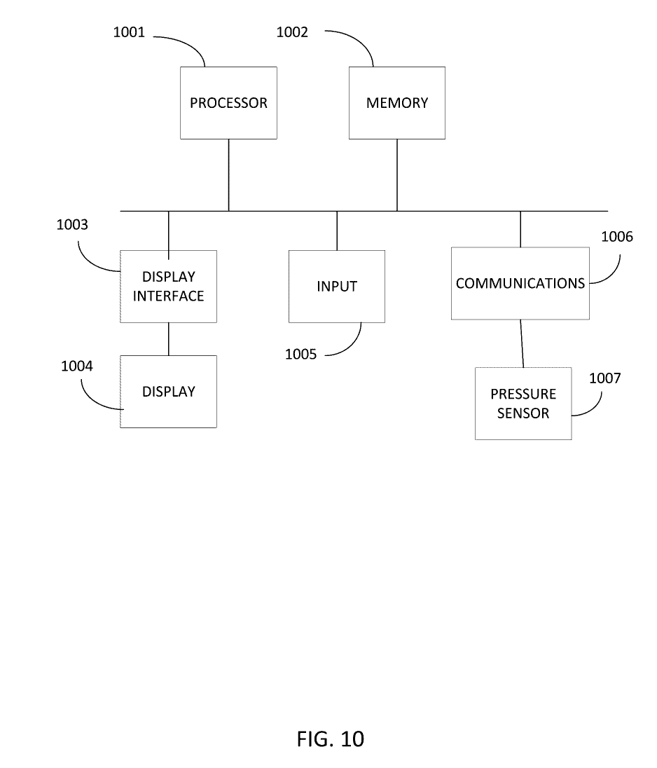

The calculations listed above may be implemented by one or more computing devices that implement computer-readable instructions. Referring to FIG. 10, a computing device will include one or more processing devices 1001 capable of performing calculations and logic operations required to execute a program. Examples include personal computers, laptop computers, tablet computing devices, and medical devices having processors.

Unless specifically stated otherwise, the terms “processor” and “processing device” are intended to refer to embodiments that require a single processor a single device, as well as to embodiments in which a group of processors collectively perform a function or process.

The computing device will also include or have access to a memory 1002. The terms “memory,” “computer-readable medium” and “data store” each refer to a non-transitory device on which computer-readable data, programming instructions or both are stored. Read only memory (ROM) and random access memory (RAM) constitute examples of non-transitory computer-readable storage media on which the programming instructions and/or data may be stored. Other examples include firmware, hard drives, flash drives, solid state drives and the like. Programming instructions, data and modules may be included on a single memory device, or distributed across multiple memory devices. When this document uses terms such as “computer-readable memory” and “memory device,” it is intended to include single-device embodiments, multiple device embodiments in which various data and/or instructions are stored on a set of devices, and embodiments with multiple memory sectors of one or more devices.

An optional display interface 1003 may permit information to be displayed on a display device 1004 in visual, graphic or alphanumeric format. Communication with external devices, such as a printing device, may occur using various communication ports. A communication port may be attached to a communications network, such as the Internet or an intranet. Or it may include a transmitter that transmits data via a wireless data network or near field communication network.

The hardware may also include an interface that allows for receipt of data from an input device 1005 such as a keyboard, mouse, a joystick, a touch screen, a remote control, a pointing device, a video input device and/or an audio input device.

The hardware also may include a communication device 1006 such as an input port or wireless transceiver for receiving data from an external data collection source, such as a pressure sensing device 1007 as described above.

The features and functions disclosed above, as well as alternatives, may be combined into many other different systems or applications. Various presently unforeseen or unanticipated alternatives, modifications, variations or improvements may be made by those skilled in the art, each of which is also intended to be encompassed by the disclosed embodiments.

1. A method of determining ventricular ejection fraction of a patient, the method comprising:
by a pressure sensing device, capturing pulmonary arterial pressure data for a patient over a period of time; and
by a processing device, implementing programming instructions that are configured to cause the processing device to:
receive the pressure data captured by the pressure sensing device;
generate a first time-resolved pressure curve that comprises the pressure data;
displace the pressure values of the first time-resolved curve at least one time point and subtract the displaced pressure values from the received pressure data to form a second time-resolved pressure curve so that the second time-resolved pressure curve has two or more distinct pulses from which an initial pulse may be isolated and an area may be calculated;
determine an average pressure by averaging the pressure data of the first time-resolved pressure curve over a cardiac cycle of data;
determine a cardiac chamber stroke volume for the patient;
use the determined cardiac chamber stroke volume and determined average pressure to determine an ejection fraction for the patient; and
output a report of the ejection fraction.

2. The method of claim 1, wherein determining the ejection fraction is also based on a slope of a rise in pressure during systolic contraction and the average pressure.

3. The method of claim 1, wherein determining the ejection fraction comprises applying the following equation:
EF=(SV*Emax)/(mean P*(ΔP+SV*ΔP));
wherein:
EF is the ejection fraction,
SV is the stroke volume,
Emax is a slope of a rise in pressure during systolic contraction,
mean P is the average pressure, and
ΔP is a difference between an end systolic pressure and an end diastolic pressure as determined in the pressure data.

4. The method of claim 1, wherein determining the stroke volume comprises:
determining an area under a first pulse of the first curve;
multiplying the area by a constant to yield a result; and
dividing the result by the average pressure.

5. The method of claim 1, further comprising, by the processing device:
performing a calibration step by:
using data received from an imaging modality, a flow based measurement, or other measurement means to measure a cardiac chamber stroke volume, and
using the measured cardiac chamber stroke volume to calculate the constant.

6. The method of claim 4 wherein the constant is 2.37.

7. The method of claim 1, further comprising, by the processing device, implementing programming instructions that are configured to cause the processing device to:
determine an additional cardiac chamber stroke volume for an additional time period using an additional pressure waveform for the additional time period, and the constant; and
use the additional cardiac chamber stroke volume to determine an additional ejection fraction of the patient.

8. The method of claim 1 wherein determining the cardiac chamber stroke volume comprises calculating the difference in oxygen concentration between an arterial and venous blood supply and a total oxygen consumption per minute.

9. The method of claim 1 wherein determining the cardiac chamber stroke volume comprises injecting a measured volume of cooled liquid at a measured temperature into a right atrium; and calculating a carbon monoxide level by an amount of heat lost.

10. A system for determining ventricular ejection fraction of a patient, the system comprising:
a pressure sensing device for capturing pulmonary arterial pressure data for a patient over a period of time;
a processing device; and
a memory device containing programming instructions configured to cause the processing device to:
receive the pressure data captured by the pressure sensing device,
generate a first time-resolved pressure curve that comprises the pressure data,
displace the pressure values of the first time-resolved curve at least one time point and subtract the displaced pressure values from the received pressure data to form a second time-resolved pressure curve so that the second time-resolved pressure curve has two or more distinct pulses from which an initial pulse may be isolated and an area may be calculated,
determine an average pressure by averaging the pressure data of the first time-resolved pressure curve over a cardiac cycle of data,
determine a cardiac chamber stroke volume for the patient,
use the determined cardiac chamber stroke volume and determined average pressure to determine an ejection fraction for the patient, and
output a report of the ejection fraction.

11. The system of claim 10, wherein the programming instructions are also configured to instruct the processing device to determine the ejection fraction based on a slope of a rise in pressure during systolic contraction and the average pressure.

12. The system of claim 10, wherein the programming instructions are also configured to instruct the processing device to determine the ejection fraction by applying the following equation:
EF=(SV*Emax)/(mean P*(ΔP+SV*ΔP));
wherein:
EF is the ejection fraction,
SV is the stroke volume,
Emax is a slope of a rise in pressure during systolic contraction,
mean P is the average pressure, and
ΔP is a difference between an end systolic pressure and an end diastolic pressure as determined in the pressure data.

13. The system of claim 10, wherein the programming instructions are also configured to instruct the processing device to determine the stroke volume by:
determining an area under a first pulse of the first curve;
multiplying the area by a constant to yield a result; and
dividing the result by the average pressure.

14. The system of claim 10, wherein the programming instructions are also configured to instruct the processing device to perform a calibration step by using data received from an imaging modality, a flow based measurement, or other measurement means to measure a cardiac chamber stroke volume, and using the measured cardiac chamber stroke volume to calculate the constant.

15. The system of claim 13, wherein the constant is 2.37.

16. The system of claim 10, wherein the programming instructions are also configured to instruct the processing device to:
determine an additional cardiac chamber stroke volume for an additional time period using an additional pressure waveform for the additional time period, and the constant; and
use the additional cardiac chamber stroke volume to determine an additional ejection fraction of the patient.

17. The system of claim 10 wherein the programming instructions are also configured to instruct the processing device to determine the cardiac chamber stroke volume by calculating the difference in oxygen concentration between an arterial and venous blood supply and a total oxygen consumption per minute.

18. The system of claim 10 wherein the programming instructions are also configured to instruct the processing device to determine the cardiac chamber stroke volume by calculating a carbon monoxide level by an amount of heat lost from an injected measured volume of cooled liquid.

19. The system of claim 10 wherein the pressure sensor includes an inductive-capacitive (“LC”) resonant circuit having a variable capacitor.
